(19) United States
(12) Patent Application Publication
(10) Pub. No.: US 2006/0167238 A1
(43) Pub. Date: Jul. 27, 2006

(54) Gene expression using metal ion dependent repressor/operator tandems

(75) Inventors: John R Murphy, Boston, MA (US); Robert J Harrison, Medfield, MA (US); Johanna Vanderspek, Worcester, MA (US); John Love, Pittsburgh, PA (US); Lori Bergeron, Ripon, WI (US)

Correspondence Address: LERNER, DAVID, LITTENBERG,;KRUMHOLZ & MENTLIK, 600 SOUTH AVENUE WEST, WESTFIELD, NJ, 07090, US

(73) Assignees: ADVANCED MICROBIAL SOLUTIONS CORPORATION, CAMBRIDGE, MA (US); BOSTON MEDICAL CENTER CORPORATION, BOSTON, MA (US)

(21) Appl. No.: 10/515,413

(22) PCT Filed: May 22, 2003
(86) PCT No.: PCT/US03/16187

§ 371 Date: Sep. 12, 2005

Related U.S. Application Data
(60) Provisional application No. 60/382,520, filed on May 22, 2002.

(51) Int. Cl. C12P 21/06; C07H 21/04; C12N 15/74; C12N 1/21; C07K 14/245

(52) U.S. Cl. 536/23.7; 435/69.1; 435/252.33; 435/488; 530/350

(57) ABSTRACT

Disclosed are nucleic acid circuits and methods of using same to make proteins.

### CROSS REFERENCE TO RELATED APPLICATIONS

The present application is a national phase entry under 35 U.S.C. § 371 of International Application PCT/US2003/016187, filed May 22, 2003, published in English, which claims benefit of U.S. Provisional Patent Application 60/382,520, filed May 22, 2002. The disclosures of all of said applications are incorporated by reference herein.

### STATEMENT REGARDING FEDERALLY SPONSORED RESEARCH

This invention was made with Government Support under Contract Number AI 21628 awarded by the National Institutes of Health. The Government has certain rights in the invention.

### BACKGROUND OF THE INVENTION

I. Recombinant Expression Systems

The advent of recombinant DNA technology has made it possible to express foreign proteins in bacteria. However, in order to make the use of recombinant DNA technology commercially practical, it is necessary to obtain high level expression of the foreign proteins. Strong promoters are typically employed to drive expression of a foreign protein which has been cloned in a multicopy expression vector (Queen, C., J. Mol. Appl. Genet. 2:1-10 (1983)). Subsequent improvements in expression vector systems have demonstrated the utility of additional factors which can increase the expression level of a given foreign protein. These factors can be loosely categorized as those dealing either with the genetics of the system or the physiology of the system. The genetics of the system principally involves the promoters, operators, ribosome binding sites and terminators. The physiology of the system involves both the micro and macrophysiology of the system under the growth and expression conditions employed whether in small scale or in large scale fermentation conditions. Salient features typically considered are discussed below.

II. Promoters and Operators

In whole cell bacterial and yeast expression, the genetic requirements for high level expression of a foreign protein start with the promoter. The promoter must be strong—that is, it must be capable of subverting a significant portion of the host cells transcriptional and translational apparatus towards initiating the synthesis of a large amount of mRNA and protein from the gene of interest. Ideally, the promoter must be well regulated since the expression of foreign proteins, often even at low levels, may alter microbial physiology of the host cell causing metabolic stress, or may be lethal to the host. This metabolic stress can lead to selective pressure which impedes the rapid growth of the culture to a high density (Remaut, E. et al., Gene 15:81-93 (1983); Remaut, E. et al., Nucl. Acids Res. 11:4677-4688 (1983); and Brosius, J., Gene 27:161-172 (1984)). Even in well-characterized stable [e.g., recombination deficient production strains of common bacteria such as E. coli] expression strains, this selective pressure can cause gene rearrangements, point mutations in promoters, or changes in cell physiology that result in inconsistent, reduced yields or the inability to produce the molecule of interest. Hence, a tightly regulated promoter capable of initiating rapid mRNA synthesis at high levels upon induction of recombinant gene expression has broad utility as a component of foreign gene expression systems.

Expression is dependent on the interaction of RNA polymerases with the operator/promoter sequences of the foreign gene. These interactions produce mRNA transcripts which contain further genetic signals, such as the ribosome binding site (RBS), which are involved in the translation of mRNA into protein. Phage lambda contains two promoters (lambda P_{R} and lambda P_{L}) that have been used extensively in foreign gene expression systems. Each of these promoters can synthesize a high level of mRNA. These promoters are controlled by the binding of a repressor, cI, to their respective operators (O_{R1}, O_{R2}, O_{R3}, and O_{L1}, O_{L2}, O_{L3}) which block mRNA synthesis by inhibiting the binding of RNA polymerase. The disruption of operator-repressor binding results in derepression and the synthesis of mRNA by DNA dependent RNA polymerase. Thus the phage lambda cI repressor operator circuit comprises a “genetic switch” which is suitable for the assembly of protein expression vectors.

To date, some of the most efficiently controlled promoters are obtained from mutants of phage lambda. For example, the lambda cI857 mutant contains a temperature sensitive cI repressor which is inactive at 42° C. Lambda P_{R} or lambda P_{L} promoters controlled by binding of the cI857 repressor to the lambda P_{R} or lambda P_{L} operators remain repressed at temperatures between 28° C. to 30° C. However, at 42° C., the unstable cI857 repressor no longer binds to the lambda P_{R} or lambda P_{L} operators, thereby causing derepression of mRNA synthesis (Isaacs, L. N. et al., J. Mol. Biol. 13:963-967 (1965); and Lieb, M., J. Mol. Biol. 16:149-163 (1966)).

Since there is very little detectable mRNA at 30° C. (Isaacs, L. N. et al., J. Mol. Biol. 13:963-967 (1965); and Lieb, M., J. Mol. Biol. 16:149-163 (1966)), foreign gene expression systems using the cI857 repressor to control the lambda P_{R} and lambda P_{L} promoters have been generally recognized as superior to foreign gene expression systems which employ the lac promoter (Remaut, E. et al., Nucl. Acids Res. 11:4677-4688 (1983); and Bachman, K. et al., Proc. Natl. Acad. Sci. USA 73:4174-4178 (1976)) or the trp promoter (Queen, C., J. Mol. Appl. Genet. 2:1-10 (1983)) which initiate the synthesis of mRNA at generally lower rates/levels than do the lambda promoters.

III. Hybrid Promoters

Several hybrid promoters, having strong promoter sequences from one naturally occurring gene and a non-native operator from a different gene or operon, have been described. To enhance the efficiency of the rate of transcription and translation of foreign genes, the tac promoter was assembled. Tac is a fusion of the trp promoter and lac UV5 promoter, wherein the DNA sequences 5′ to position −20 with respect to the transcriptional start site are derived from the trp promoter and the DNA sequences 3′ to position −20 with respect to the transcriptional start site are derived from the lac UV5 promoter (DeBoer, H. A. et al., Proc. Natl. Acad. Sci. USA 80:21-25 (1983)); the O_{L}/P_{R} promoter which results from the fusion of the lambda O_{L} operators to the lambda P_{R} promoter at a common HincII site located in their respective −35 regions. The resultant hybrid promoter comprises the operator region of lambda O_{L}, including the O_{L}−35 region, followed by the lambda P_{R} promoter sequences starting at the lambda P_{R}−35 region and proceeding downstream through its −10 region, continuing through the mRNA initiation site and including the lambda P_{R} RBS (U.S. Pat. No. 4,551,433). This promoter is activated by raising the culture temperature. Repression at 30° C. is maintained by the lambda cIl857 repressor binding to the lambda O_{L2} and O_{L3} repressor sites in the lambda O_{L} operator. Raising the temperature to 42° C. inactivates the lambda repressor (Isaacs, L. N. et al., J. Mol. Biol. 13:963-967 (1965)), allowing binding of RNA polymerase and mRNA synthesis (U.S. Pat. No. 4,868,125); and the let promoter which results from the fusion of the lambda P_{L} operator to a portion of the trp promoter, wherein the trp promoter extending 5′ from the mRNA initiation site, including the trp operator and the trp −35 region, is fused to the lambda P_{L} operator using the HincII site located in the −35 region of the lambda P_{L} operator (Nishi, T. et al., Gene 44: 29-36 (1986)).

The tac promoter has been shown to direct expression of some genes, such as human growth hormone and galactokinase, at reasonable levels in shaker flasks (DeBoer, H. A. et al., Proc. Natl. Acad. Sci. USA 80:21-25 (1983)). However, the use of the tac promoter is limiting in that it cannot be completely repressed unless it is used in a strain which over-produces the lac repressor. This genetic construction results in a genetic switch which does not allow full induction of the tac promoter and high levels of recombinant gene expression due to the high steady state levels of lac repressor present (Remaut, E. et al., Gene 15:81-93 (1981); and Backman, K. et al., Proc. Natl. Acad. Sci. USA 73:4174-4178 (1976)). These problems are not unique to the tac promoter and present a serious disadvantage when the expression of a foreign gene has a toxic effect on host cell physiology, as in the case of human proinsulin and DNA polymerase I (Brosius, J., Gene 27:161-172 (1984); Remaut, E. et al., Nucl. Acids Res. 11:4677-4688 (1983); Hallwell, R. A. et al., Gene 9:27-47 (1980); Tacon, W. et al., Mol. Gen. Genet. 177:427-438 (1980); Queen, C., J. Mol. Appl. Genet. 2:1-10 (1983); Remaut, E. et al., Gene 15:81-93 (1981); and Kelly, W. S. et al., Proc. Natl. Acad. Sci. USA 74:5632-5636 (1977)).

### SUMMARY OF THE INVENTION

The unwanted or mistimed expression of foreign proteins during the production of recombinant proteins in E. coli or other prokaryotic cell systems can have detrimental effects on the host organism. The majority of regulated gene expression systems which seek tightly controlled recombinant protein expression exhibit undesirable “leaky expression”. The present invention describes genetic circuitry using metal ion dependent repressor/operator tandems and their methods of use to circumvent this problem.

A first aspect of the present invention relates to a composition of matter, comprising: a first nucleic acid encoding a repressor protein activated by a metal ion activator, and a second nucleic acid comprising a promoter, an operator sequence and a multiple cloning site for introduction of at least one structural gene in operable association with each other, such that in the presence of the metal ion, activated repressor binds said operator and inhibits expression of the structural gene.

Another aspect of the present invention is directed to a composition of matter, comprising: a first nucleic acid encoding a repressor protein activated by a metal ion activator, and a second nucleic acid comprising a promoter, an operator sequence and at least one structural gene in operable association with each other, such that in the presence of the metal ion, activated repressor binds said operator and inhibits expression of said at least one structural gene. In some preferred embodiments, this aspect of the present invention is directed to a composition, comprising: an E. coli cell comprising a first non-native nucleic acid encoding DtxR or a mutant or a homolog thereof, and a second nucleic acid comprising a promoter, an operator that binds said DtxR or mutant or homolog thereof, and at least one structural gene, in operable association with each other such that in the presence of a metal ion activator, said DtxR or mutant or homolog thereof is activated and binds said operator and inhibits expression of said at least one structural gene.

A further aspect of the present invention is directed to methods for making proteins. Some embodiments are directed to a method for producing a protein, comprising: transforming a prokaryotic cell with a first nucleic acid encoding a repressor protein, and a second nucleic acid comprising a second nucleic acid comprising a promoter, an operator and a structural gene encoding the protein, each in operable association with each other; growing transformed prokaryotic cells in a medium comprising a metal ion activator of the repressor, wherein activated repressor binds the operator; reducing activity of the activator to cause expression of the structural gene; and optionally isolating the protein from the prokaryotic cell or the medium. Some preferred embodiments of the methods are directed to a method for producing a protein, comprising: transforming an E. coli cell with a first nucleic acid encoding a metal ion-dependent repressor which is DtxR or a homolog thereof, and a second nucleic acid comprising a promoter, an operator that binds the DtxR or homolog thereof, and a structural gene encoding the protein, each in operable association with each other; growing transformed E. coli cells in a medium comprising a metal ion that activates the repressor, wherein activated repressor binds the operator; reducing free metal ion in the medium to cause expression the structural gene; and optionally isolating the protein from the E. coli or the medium.

In yet other embodiments, the genetic circuitry of the present invention may be utilized in a non-cellular system or environment in which to make one or more proteins. These aspects are directed to a method for producing a protein, comprising: preparing a system comprising a first nucleic acid encoding a repressor protein, and a second nucleic acid comprising a second nucleic acid comprising a promoter, an operator and a structural gene encoding the protein, each in operable association with each other; wherein said system further comprises a metal ion activator of the repressor, wherein activated repressor binds the operator; reducing activity of the activator to cause expression of the structural gene; and optionally, isolating the protein from the system.

### BRIEF DESCRIPTION OF THE DRAWINGS

FIGS. 1 A-D are DNA sequences and a plasmid map. DNA sequences of tac promoter (tacp) (FIG. 1A)(SEQ ID NO:10), diphtheria toxin promoter/operator (toxPO) (FIG. 1B)(SEQ ID NO:l1), and hybrid tacPtoxO promoter-operator (FIG. 1C)(SEQ ID NO:12). Bold letters indicate nucleotides that comprise the two inverted repeats of toxO. “−35” and “−10” sequences are underlined and labeled. The unique KpnI site engineered in tacPtoxO is in lower-case letters. FIG. 1D shows a plasmid map of pJL1.

FIGS. 2 A and B are bar graphs showing results of β-galactosidase assays comparing expression of lacZ in two reporter strains. DH5α/λJXRS45-toxPO-lacZ (dark grey) or DH5α/λRS45-tacPtoxO-lacZ (stippled) was transformed with pROM or pROM-DtxR. Cultures were grown in the absence (FIG. 2A) or presence (FIG. 2B) of 300 μM 2,2′-dipyridyl (DP).

FIG. 3 is a bar graph showing results of in vitro expression of luciferase in S30 extracts of E. coli programmed with pBEST, pJL1, and pJL12 in coupled in vitro transcription/translation assays. The tacPtoxO hybrid promoter/operator in pJL1 produced a luciferase signal comparable to that from tacP in pBEST. In contrast, pJL12, in which toxPO controlled luc expression, showed minimal luciferase production. Data shown are means of at least three experiments with their standard deviations.

FIG. 4 is a bar graph showing results of coupled in vitro transcription/translation assays. 1.0 μg of pJL1 was incubated with varying amounts of purified DtxR. Repression of luciferase expression occurred with increasing DtxR concentrations. This repression was completely relieved by the addition of the transition metal cation chelator, 2,2′-dipyridyl (DP). Data presented are the means of at least three experiments with their standard deviations.

FIG. 5 shows an alignment of the Enterococcus faecalis ABC operon promoter region. Comparison of the efa box with the corresponding permeases from related microbes. Efa, E. faecalis (SEQ ID NO:13). ScaC, Streptococcus gordonii (SEQ ID NO:14) (6), sloA, Streptococcus mutans (SEQ ID NO:15) (7), psaB, S. pneumoniae (SEQ ID NO:16) (described in (6). The consensus for the DtxR box is included (SEQ ID NO:17). Arrows indicate AT rich palindromes.

FIG. 6A is a bar graph showing activity of the efaA promoter operator region in E.coli in the presence of EfaR and Mn. Beta-galactosidase activity from efaA promoter operator exceeded that for tacP but was not completely silenced by EfaR and metal ion in the native configuration (SEQ ID NOS: 13, 18 and 19). FIG. 6B a photograph of an EMSA analysis of EfaR binding to [³²P]-labeled efa ABC box, wherein: lane 1: probe alone; lane 2, Probe plus EfaR; and lane 3, probe plus EfaR in the presence of 2,2′-dipyridyl.

FIG. 7 is a photograph showing that SirR binds to the sitABC box and DtxR, EfaR, and SirR bind to the toxP/O. Lanes 6 and 7 show SirR binding to the toxP/O and the sensitivity of the complex to metal ion depletion in lane 7 (SEQ ID NO:20).

FIG. 8 shows nucleic acid sequences of various hybrid promoter/operator sequences useful in the present invention (SEQ ID NOS: 21-25).

FIG. 9 is a photograph of a Western blot analysis of DAB₃₈₉ expression from ptacP/toxO expression vectors employing variants of DtxR. Wild type DtxR and the metal ion insensitive mutant DtxR E175I each repressed DAB₃₈₉ expression (lanes marked ‘0’ above figure) until induction by metal ion chelation (lanes marked 200, 300, and 400 for concentration of DP added). In contrast, the defective DtxR H106A mutant was unable to regulate expression of DAB389 from ptacP/toxO (last two lanes at right, marked ‘0’ and ‘400’ above figure).

FIG. 10 is a bar graph showing results of induction of DAB₃₈₉IL7 expression in E. coli HMS 174 DE3. The growth of E. coli carrying ptacP/toxO based vectors was rapid and robust even following de-repression with DP. Induction was initiated by the addition of DP to 300 uM at 120 minutes. The culture continued to grow and was harvested 2.5 hours later. In contrast, cultures expressing DAB₃₈₉IL7 from a pET vector grew more slowly and never reached an OD of greater than 1.0. These cultures were induced with lmM IPTG at 300 minutes and were harvested 3.0 hours later. Both inductions yielded protein which cross-reacted with anti-diphtheria toxin and anti-IL7 antibodies.

### DETAILED DESCRIPTON OF THE INVENTION

The present invention relates to the controlled expression of recombinant proteins. In the preferred embodiment the expression is achieved in a prokaryotic organism such as E. coli. Expression is typically directed by promoter sequences identified from E. coli and controlled by limiting the amount of an inducer or specific polymerase. Inducers disrupt the interaction between a negative regulator, repressor, which in the un-induced state serves to limit protein expression. To date these systems all permit read through or leaky low level expression of the recombinant protein even in the absence of the inducer or requisite polymerase. Since foreign protein expression is often toxic to the host organism, the recombinant E. coli tend to select against the desired recombinant genes, induce stress responses which degrade the desired product or yield only small amounts of the desired recombinant protein.

The present invention provides for a combination of genetic elements arranged for use in methods of recombinant gene expression. The genetic elements, the repressors employed and the arrangement of the genetic elements more completely silences recombinant gene expression in the un-induced state, thereby limiting selective pressure, against the recombinant gene of interest, and other unwanted responses, thereby enhancing the yield of the desired product.

The present invention provides a first nucleic acid encoding a metal ion-dependent repressor (a repressor gene) and a second nucleic acid comprising a promoter, an operator sequence and a multiple cloning site for introduction of at least one structural gene of interest, and/or the structural gene, per se, all in operable association with each other. In the presence of the free metal ion (e.g., a transition metal ion such as iron or nickel), the expression product of the first gene is activated by the transition metal ions and binds the operator silencing or preventing expression of the structural gene.

In preferred embodiments, the repressor encodes DtxR or a mutant (e.g., a functional fragment or variant) or a homolog (all of which may be collectively referred to as “a DtxR protein”). DtxR is an iron-dependent DNA-binding protein having a deduced molecular weight of 25,316 and which functions as a global regulatory element for a variety of genes on the C. diphtheriae chromosome. (See Tao et al., Proc. Natl. Acad. Sci. USA 89:5897-5901 (1992); Schmitt et al., Infect. Immun. 59:1899-1904 (1994).) For example, DtxR regulates the expression of the diphtheria toxin structural gene (tox) in a family of closely related Corynebacteriophages. The dtxR gene has been cloned and sequenced in E. coli and its DNA and amino acid sequences have been reported. (See Boyd et al., Proc. Natl. Acad. Sci. USA 87:5968-5972 (1990); Schmitt et al., supra.) DtxR is activated by divalent transition metal ions (e.g., iron). Once activated, it specifically binds the diphtheria tox operator and other related palindromic DNA targets. (See Ding et al., Nature Struct. Biol. 3(4):382-387 (1996); Schiering et al. Proc. Natl. Acad. Sci. USA 92:9843-9850 (1995); White et al., Nature 394:502-506 (1998).)

Mutants of DtxR, when activated, retain their binding activity to the tox operator (or a functional fragment thereof) and/or the DtxR consensus binding sequence. DtxR mutants such as fragments and variants can be identified by standard techniques such as mutagenesis. It has been reported that the Cys102 residue in DtxR is important in binding the tox operator and substitutions with amino acids other than Asp abolish binding activity. (Tao et al., Proc. Natl. Acad. Sci. USA 90:8524-8528 (1993).) Other variants are disclosed in Tao et al., Mol. Microb. 14(2):191-197 (1994). Tao discloses that some dtxR alleles have different amino acid sequences, e.g., the dtxR allele from strain 1030(-) of C. diphtheriae was found to carry six amino acid substitutions in the C-termninal region, none of which affected the iron-dependent regulatory activity of DtxR (1030) (Tao 1994). (See also Boyd et al., J. Bacteriol. 174:1268-1272 (1992) and Schmitt et al., Infect. Immun. 59:3903-3908 (1991).) Examples of suitable DtxR mutants suitable for use in the present invention include DtxR E175I and DtxR E175K.

Many other bacterial species employ regulatory circuits and repressor proteins that exhibit high degrees of sequence similarity to DtxR. Thus, DtxR homologs may also be employed in the methods of the present invention. Iron dependent regulator (IdeR), isolated from Mycobacterium tuberculosis, has been found to share 60% homology or sequence similarity with DtxR. (See Schmitt et al., Infect Immun. 63(11):4284-4289 (1995; see also Doukhan et al., Gene 165(1):67-70 (1995), which reports and references DtxR homologs in Mycobacterium smegmatis and Mycobacterium leprae.) DtxR homologs have been cloned in other gram-positive organisms including Brevibacterium lactofermentum and Streptomyces lividans. (See Oguiza, et al., J. Bacteriol. 177(2):465-467 (1995); Guinter, et al., J. Bacteriol. 175:3295-3302 (1993); and Schmitt, et al., Infect. Immun. 63:4284-4289 (1995).) Staphylococcal iron regulated repressor (SirR), native to Staphylococcus epidermitis, is another known DtxR homolog. EfaR, the repressor native to Enterococcus faecalis, is yet another DtxR homolog suitable for use in the present invention. These proteins bear a common feature—they share a remarkably high sequence similarity in the respective N-terminal 139 amino acid regions, especially those amino acids involved in DNA recognition and transition metal ion co-ordination. In addition to DtxR homologs, DtxR sensitive promoters and/or genes involved in a variety of cellular activities have been cloned from C. diphtheriae chromosomal libraries. (See Schmitt et al., J. Bacteriol. 176:1141-1149 (1994), and Schmitt, J. Bacteriol. 179:838-845 (1997).)

A collection of accession numbers for sequences that are homologous to DtxR, or contain the consensus toxO sequence, is presented below. (See http://www.ncbi.nlm.nih.gov/BLAST and http://www.ncbi.nlm.nih.gov/unfinishedgenomes.html. (See also, Altschul, et al., J. Mol. Biol. 215:403-410 (1990); Gish, et al., Nature Genet. 3:266-272 (1993); Madden, et al., Meth. Enzymol. 266:131-141 (1996); Altschul, et al., Nucleic Acids Res. .25:3389-3402 (1997); and Zhang, et al., Genome Res. 7:649-656 (1997).) This degree of sequence similarity in the homologs and the distribution of the operator sequence indicates that the iron regulatory pathway that employs the DtxR-family of repressors is conserved in many important human and animal pathogens.

| Pathogenic Human/ | | | |
|---|---|---|---|
| Veterinary Applications | | Other | |
| CAA67572 | S. epidermidis * | L35906 | C. glutamicum |
| Gi 1777937 | T. pallidum | Z50048 | S. pilosus |
| CAA15583 | M. tuberculosis * | Z50049 | S. lividans |
| U14191 | M. tuberculosis * | U14190 | M. smegmatis |
| L78826 | M. leprae * | L35906 | B. lactofermentum |
| M80336 | C. diphtheriae * | | |
| M80337 | C. diphtheriae * | | |
| M34239 | C. diphtheriae * | | |
| M80338 | C. diphtheriae * | | |

* = species also contains toxO sequences

Selection of DNAs Homologous to DtxR Identifiable in Current Databases

| | | | |
|---|---|---|---|
| Gi 2622034 | M. thermoautotrophicum | Stanford 382 | S. meliloti |
| Gi 2621260 | M. thermoautotrophicum | TIGR 1280 | S. aureus |
| M50379 | M. jannaschi | OUACGT | S. pyogenes |
| Q57988 | M. jannaschi | Sanger 518 | B. bronchoseptica |
| O33812 | S. xylosus | Sanger 1765 | M. bovis * |
| Gi 264870 | A. fulgidus | Sanger 520 | B. pertusis * |
| Gi 2648555 | A. fulgidus | WUGSC | K. pneumoniea |
| Gi 2650396 | A. fulgidus | TIGR 76 | C. crescentus |
| Gi2650706 | A. fulgidus | TIGR 24 | S. putrificacieus |
| BAA79503 | A. pernix | TIGR 1351 | E. faecalis |
| AAD18491 | C. pneumoniae * | AE000783 | B. burgdorferi |
| Gi 3328463 | C. trachomatis | TIGR1313 | S. pneumoniea * |
| CAB49983.1 | P. abyssi | Snager 632 | Y. pestis |
| BAA30263 | P. horikoshi | AE000657 | A. aeolius |
| Gi 2621260 | M. thermoautotrophicum | TIGR 920 | T. ferrooxidans |
| TIGR 1752 | V. cholera | AE001439 | H. pylori |

Corresponding operator sequences may be designed on the basis of the native sequence (e.g., in the case of DtxR, toxO). Alternatively, they may differ from the native operator sequence provided that the requisite binding occurs. Examples of repressor/operator gene tandems useful in the present invention are described in U.S. Pat. No. 6,309,817, issued Oct. 30, 2001 to Murphy, et al. (and publications cited therein). Thus, in embodiments where the repressor is DtxR, a preferred operator is the natively associated tox operator, toxO, a functional fragment thereof, or a variant of a DtxR consensus binding sequence. The native tox operator (i.e., 5′-ATAATTAGGATAGCTTTACCTAATTAT-3′; SEQ ID NO:1) is a 27-base pair interrupted palindromic sequence upstream of the diphtheria tox structural gene; it features a 9-base pair inverted repeat sequence that is separated by 9-base pairs. (See Kaczorek et al., Science 221:855-858 (1983); Greenfield et al., Proc. Natl. Acad. Sci. USA 80:6853-6857 (1983); Ratti et al., Nucleic Acids Res. 11:6589-6595 (1983); and Fourel et al., Infect. Immunol. 57:3221-3225 (1989).) It overlaps both the -10 region of the tox promoter and the transcriptional start sites at −45, −40 and −39 upstream of the diphtheria toxin structural gene. (See Boyd, et al., J. Bacteriol. 170:5940-5952 (1988).) The minimal essential DNA target site, i.e., 5′-GTAGGTTAGGCTAACCTAT-3′ (SEQ ID NO:2), is a 19-base pair sequence that forms a perfect palindrome around a central C or G. It is described in Tao and Murphy, Proc. Natl. Acad. Sci. USA 91:9646-9650 (1994). In some preferred embodiments, the native promoter Pₜₒₓ is operably linked to the operator, resulting in the construct known as toxPO. Also preferred are variants of toxO based on the DtxR consensus-binding sequence (5′- ANANTTAGGNTAGNCTANNCTNNNN-3′; SEQ ID NO:3). The variants are defined by the following sequence: 5′-TWAGGTTAGSCTAACCTWA-3′ (SEQ ID NO:4). Yet other operator sequences that bind DtxR, DtxR mutants and DtxR homologs may be obtained in accordance with routine screening as illustrated in example 2 set forth in U.S. Pat. No. 6,309,817.

The genetic circuitry of the present invention may be employed in any prokaryotic cell such as a bacterium, in which they are functional, to make proteins. In preferred embodiments, the methods are practiced using E. coli as a host. Plainly, a promoter is chosen that is functional in the host of choice. In preferred embodiments of the invention, the promoter initiates high-level expression of the structural gene in the host. In the case of E. coli, preferred promoters that direct high levels of recombinant gene expression include T7 promoter, Tac promoter, and the disclosed sequences of the sit ABC promoter of Staphylococcus (e.g., aureus and epidermidis) and Enterococcus. The promoter and the operator may be native to each other (e.g., toxP/O). In addition, Examples 1 and 2 below illustrate the use of hybrid promoter/operator tandems including sequences isolated from Enterococcus faecalis designated as efaPO. The corresponding Mn²⁺ ion-dependent repressor in E. faecalis, EfaR, is a homolog of DtxR.

To construct the nucleic acids of the present invention for use in a particular host, it may be advantageous to vary the spacing between the promoter and the operator in order to determine optimal expression. This is illustrated in Example 1.

The compositions and methods of the present invention are useful to produce a wide variety of proteins. Representative proteins that can be made recombinantly using the present invention include therapeutics or targets for in vitro screening, enzymes for biocatalysis, restriction enzymes, DNA/RNA binding proteins, antigens for vaccine production, and proteins that typically are expressed at low levels due their intrinsic toxic effects on the expression host. Other bacteria besides E. coli can be used as a host system, (e.g., Bacillus).

Practice of some methods of the present invention entails transforming a prokaryotic host with the first and second nucleic acids and then growing the transformed host in culture in the presence of an activator of the repressor (e.g., a free transition metal ion) for a predetermined time, (e.g., until the cells reach a predetermined density for the intended purpose). The first and second genes may be introduced into the host together e.g., contained on the same vector, plasmid, or separately, each in accordance with standard techniques. The repressor gene may be introduced into the chromosomal DNA of the host so as to be under the control of a native promoter. Alternatively, it may be introduced into the host already functionally linked to a promoter that drives expression of the repressor gene even in the presence of the activator. The most suitable activators are metal ions; however non-metal ion activators are also suitable. In the presence of a freely available metal ion in a standard undefined microbiological or a synthetic (e.g., M9 medium) or semi-synthetic media (bhi/luria broth etc.), the metal dependent repressor is maintained in an active state so as to bind the operator sequence, silencing gene expression. When the desired cell density is attained (which for maximal expression may be determined empirically in accordance with standard techniques but generally ranges in terms of Absorbance at 590 nm (A590 nm) from about 0.4 to about 0.8) expression of the structural gene is initiated by lowering the concentration of the free metal ion. The addition of an inducer (e.g., a metal ion chelator) will accomplish this and de-repress the operator. In the case of DtxR or homologs or mutants thereof, a particularly suitable chelator is 2,2′-dipyridyl. Final concentrations of 2,2′-dipyridyl added to the growth medium generally range from about 100 to 300 μM. Under these conditions the promoter element functionally linked to the structural gene drives expression (or in the case of high expression level promoters, over-expresses) of the gene of interest. The expression product may be harvested from the cells or culture and purified in accordance with standard techniques for the given host system. Typically, the expression product is contained in an inclusion body or in the periplasmic space, but in some embodiments, it is exported to the extracellular space.

The methods of the present invention may also be practiced in non-cellular systems or environments, such as illustrated in Example 1. In such settings, transcription and translation reactions are conducted separately.

The invention will be further described by reference to the detailed examples. These examples are provided for purposes of illustration only, and are not intended to be limiting unless otherwise specified.

### SUMMARY OF EXAMPLE 1

We have constructed a novel hybrid promoter/operator-lacZ transcriptional fusion in which the “−35” and spacing of the tac promoter was fused to the “−10” and interrupted palindromic sequence of toxO. We show that the hybrid tacPtoxO is regulated by the transition metal ion-dependent DtxR and that lacZ expression is increased approximately 70-fold in the reporter strain Escherichia coli DH5α/λRS45-tacPtoxO-lacZ relative to DH5α/λRS45-toxPO-lacZ. In addition, we have constructed a transcriptional fusion between tacPtoxO and luc, pJL1. We have used pJL1 to program S30 extracts of E. coli in order to direct in vitro the coupled transcription and translation of luciferase. We demonstrate the utility of this in vitro system in providing a direct functional link between in vivo and in vitro observations with DtxR and mutants of DtxR that exhibit both high levels induced of expression and tight repression in the presence of activating metal ions.

### EXAMPLE 1

DtxR is a 226 amino acid, 26 kDa protein expressed by Corynebacterium diphtheriae. In the presence of divalent transition metal cation (notably Fe⁺², but also Ni⁺², Co⁺², Mn⁺², and Cd⁺²), DtxR undergoes structural changes, leading to dimerization (Tao & Murphy, 1992; Schmitt & Holmes, 1993; Tao et al., 1995). Two DtxR dimers bind to opposite faces of toxO, covering the “−10” sequence of toxP, and repressing transcription (White et al., 1998). Homologues of DtxR have been identified in numerous Gram-positive prokaryotes, and this family of regulatory proteins is believed to control expression of virulence determinants as well as iron uptake and storage systems in response to Fe⁺² (or other divalent transition metal cation) concentrations (Jakubovics et al., 2000; Oguiza et al., 1995; Que & Helmann, 2000; Schmitt et al., 1995).

We have constructed a unique, highly sensitive reporter gene expression system by fusing the toxO sequence with the E. coli tac promoter (tacP) to control and direct the expression of either lacZ or luc. The data presented in this example illustrate the unique sensitivity of this expression system to levels of metal ion and the utility of the hybrid tacPtoxO- promoter in regulating gene expression both in vivo and in vitro. (Ding 1996).

2. Materials and Methods

2.1 Strains and plasmids

E. coli DH5α and E. coli TOP10 were obtained from Novagen. E. coli NK7049, λRS45, and pRS551 were generously supplied by R. W. Simons. pBEST came from Promega. pET-11c was purchased originally from New England Biolabs.

2.2 Culture conditions

All bacterial cultures were grown in Luria broth media, supplemented with 100 μg/mL ampicillin and/or 50 μg/mL kanamycin. Cloning procedures used E. coli TOP10 grown overnight in LB with ampicillin at 37° C. in a Rollordrum (New Brunswick Scientific).

2.3 PCR, cloning, and related DNA techniques

Mutagenic PCR was performed according to the Quick-Change Mutagenesis protocol published by Stratagene. Mutagenic PCR reactions were digested with DpnI before transformation into E. coli TOP10. Traditional PCR amplification reactions utilized 250 ng of primer and 20 ng template. Both forms of PCR used primers synthesized by GibcoBRL, deoxynucleotides purchased from Perkin-Elmer, and reaction buffer and Pfu Turbo purchased from Stratagene.

Restriction enzymes were purchased from New England Biolabs, and digestions were done according to manufacturer's suggested protocols. The toxO sequence was synthesized as two oligonucleotides by GibcoBRL, annealed, and phosphorylated by T4 DNA kinase (New England Biolabs). DNA preparation kits from Promega were used for DNA extractions.

2.4 Production of recombinant λ phage

Work with λRS45 followed protocols outlined previously in this laboratory (Boyd et. al., 1990). All reagents and media used were prepared according to Maniatis et al., 1982.

2.5 β-galactosidase assays

Overnight cultures in LB/amp/kan were started from single colonies. For cultures grown in the presence of the chelator 2,2′-dipyridyl, solid DP was dissolved in ethanol immediately before addition, and the final concentration of DP in the culture was 300 μM. Eight mL cultures were grown overnight at 37° C. in a Rollodrum (New Brunswick Scientific) at approximately 50 rpm. The β-galactosidase assay was performed as described by Miller, 1977. Briefly, 500 μL of overnight culture was vortexed for 10 seconds with 15 μL lysis buffer (400 μL chloroform with 200 μL 10% sodium dodecylsulfate). 25 μL (DH5α/λRS45-tacPtoxO-lacZ) or 200μL (DH5α/λRS45-toxPO) was added to Z buffer to make 1.0 mL reaction mixtures. 200 μAL of o-nitrophenol β-D-galactopyranoside (ONPG, 4 mg/mL) was added, and the reactions incubated at 25° C. for up to 60 minutes. The reaction was stopped by the addition of 0.5 mL of 1.0 M sodium carbonate. Spectroscopic readings were taken both of the overnight culture and of the β-galactosidase reaction, and units of β-galactosidase were calculated as described by Miller, 1977.

2.6 Coupled in vitro transcription/translation reactions

Coupled in vitro transcription/translation reactions were carried out following the guidelines of an E. coli S30 extract system for use with circular DNA templates. (Promega) In a total volume of 35 μL, reaction buffer, amino acids, 1.0 μg of template DNA, and purified protein (if applicable) were mixed and allowed to incubate at room temperature for 10 minutes. Afterwards, 15 μL of E. coli S30 extract was added, and the reaction was incubated at 37° C. for 60 minutes. Following incubation, reactions were stopped by cooling on ice for at least 15 minutes. Reactions were diluted using luciferase dilution buffer, and luciferase activity was analyzed using a Turner Designs luminometer (TD-20/20).

3. Results

3.1 Construction of tacPtoxO Hybrid Promoter/Operator

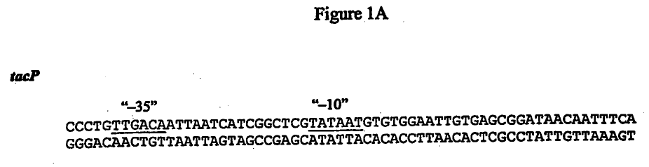

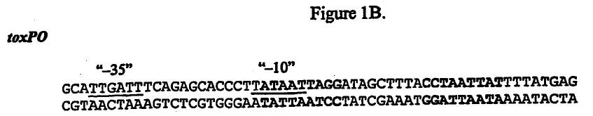

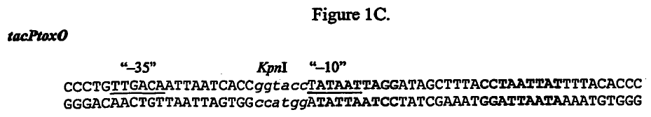

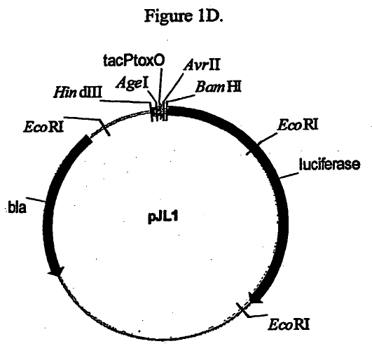

Several elements are known to affect the relative strength of promoters in E. coli. Among these, the sequences of the “−35” and “−10” regions and the intervening spacing between these two sites are particularly influential on basal level expression. Because the tac promoter has canonical “−10” and “−35” sequences as well as optimal spacing between those two elements, we chose it as the foundation for the construction of a novel tacPtoxO hybrid promoter/operator. Starting with pBEST, we introduced the toxO sequence into tacP, while maintaining the “−10” and “−35” sequences and spacing of tacP, producing pJLl (FIGS. 1A-D). In this construct, the toxO sequence overlaps the “−10” sequence, as it does in the native toxPO, and the intervening sequence between the “−35” and “−10” is the same as in tacP.

Purified DtxR binds toxPO and tacPtoxO as determined by an electrophoretic mobility shift assay using appropriate DNA probes. In contrast, DtxR was unable to shift the tacP probe. Thus, DtxR was able to bind its target operator in both its native promoter and in the new hybrid promoter (Love et al., 2002).

3.2 Construction of an Improved Assay System for in vivo DtxR Activity

Previous in vivo work with DtxR utilized an E. coli reporter strain which had been lysogenized with recombinant X phage carrying a toxPO-lacZ transcriptional fusion (E. coli DH5α/λRS45-toxPO-lacZ) (Boyd et al., 1990).

The tacPtoxO sequence was PCR amplified from pJLI with primers including EcoRI and BamHI sites. The product was cut and ligated into similarly digest pRS551-toxPO-lacZ, to yield pRS551-tacPtoxO-lacZ. Following transformation of E. coli TOP 10, clones were isolated and plasmid DNA sequenced to confirm fidelity. The pRS551-tacPtoxO-lacZ was then introduced into recA⁺E. coli NK7047. Transformants were isolated for resistance to ampicillin and subsequently infected with λRS45. Phage were collected and plated on lawns of E. coli NK7047. Recombinant phage identified by blue plaques on LB agar plates containing X-gal were picked, and purified by repeated cycles of phage plating on NK7947 lawns to isolate the recombinant clone λRS45 tacPtoxO-lacZ. The cloned recombinant phage was then used to infect E. coli DH5α. A single kanamycin-resistant, blue colony was isolated, re-streaked multiple times on LB-kanamycin agar to ensure purity, and then designated E. coli DH5α/λRS45-tacPtoxO-lacZ.

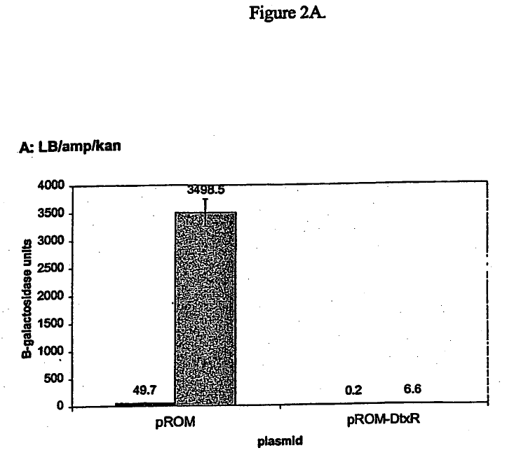

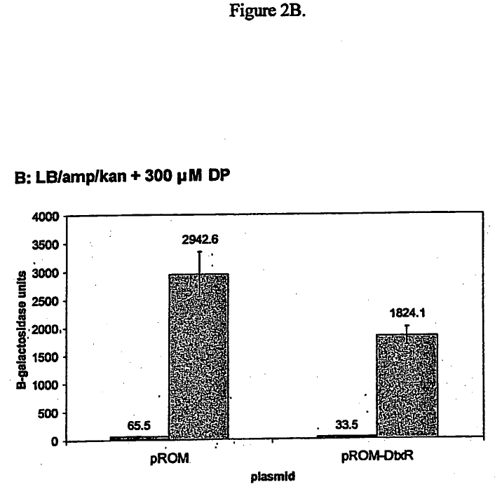

The T7 promoter of pET11c was replaced with the native promoter of dtxR, producing pROM. The dtxR structural gene was then cloned downstream of this promoter, yielding pROM-DtxR. Each of these plasmid constructs was then independently transformed into both competent DH5α/λRS45-tacPtoxO-lacZ and DH5α/λRS45-toxPO-lacZ. Following overnight incubation, β-galactosidase assays were performed on cultures of each transformant, both in the absence and presence of the divalent transition metal cation chelator 2,2′-dipyridyl. As shown in FIG. 2A, both strains demonstrated complete DtxR-mediated repression of lacZ, and as expected this repression was cation-dependent, as measured by inactivation by the chelator 2,2′-dipyridyl. The hybrid promoter operator, tacPtoxO strain demonstrates seventy times more β-galactosidase activity than the toxPO transcriptional fusion. (FIG. 2B.) Significantly, the ratio of DtxR-repression to background is roughly equivalent for both strains, demonstrating the ability of the DtxR-repressor toxO-operator circuit to repress the more potent tac promoter yielding an increase in the amplitude of the output expression signal.

3.3 Function of the tacPtoxO Regulatory Circuit in in vitro Expression Studies

An extremely sensitive expression system using a coupled in vitro transcription/translation assay using S30 extracts of E. coli to drive gene expression from exogenous plasmid DNA. As described above, pJLl was constructed from pBEST. In pJL1, luc is regulated by the hybrid tacPtoxO sequence, while pBEST has the original tac promoter upstream of luc. For comparison, we also constructed pJL12, in which toxPO directs expression of luc.

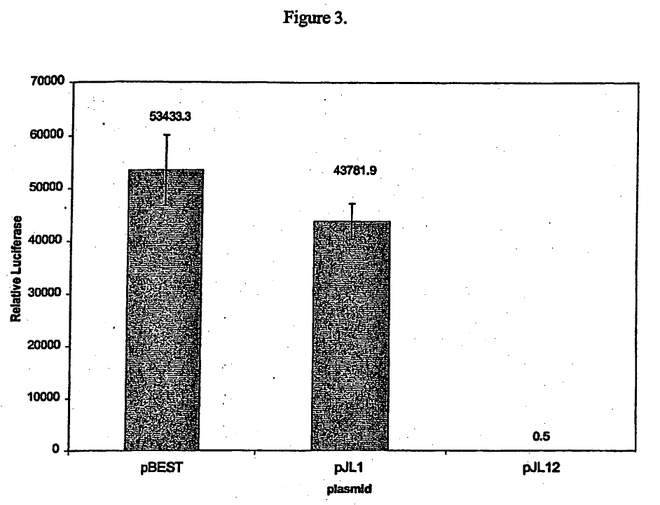

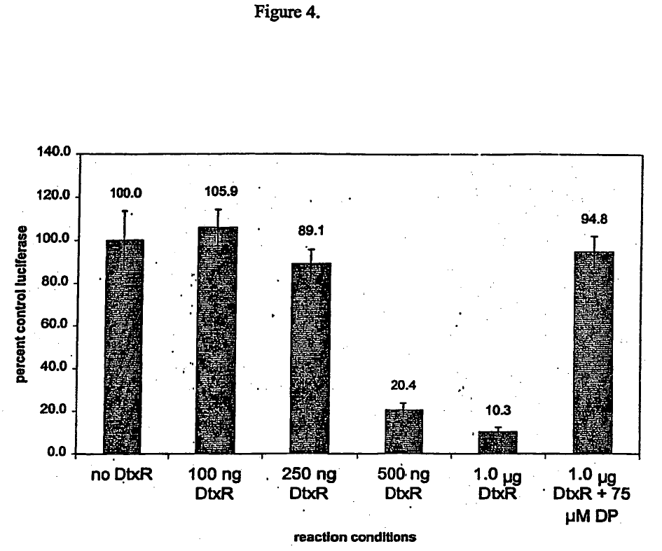

As shown in FIG. 3, all three plasmids direct the in vitro expression of luciferase when added to S30 extracts of E. coli. As seen in vivo, the native toxPO on pJLI2 was a weak promoter of luc expression. In contrast, both pBEST and pJLI demonstrated high luciferase production. Importantly, the in vitro production of luciferase from pJLI is completely repressed by the addition of purified DtxR in a dose-dependent manner (FIG. 4) as expected. Moreover, DtxR-mediated repression is cation-dependent: the addition of 2,2′-dipyridyl to the coupled in vitro transcription/translation reaction results in de-repression of luc expression. Since addition of 2,2′-dipyridyl alone minimally decreases the background level of luc expression, reactions that differ only in the presence or absence of DtxR are compared, and luciferase expression is presented as percent control expression. The addition of 100 μM NiCl₂ with 1.0 μg DtxR did not alter luciferase levels, suggesting that DtxR, and not activating cation, limits repression in these reactions (data not shown).

When examining individual mutations of DtxR, the relative in vitro expression of luciferase can be compared to relate their repressor activities. The S30 in vitro coupled transcription/translation assay is the first direct in vitro assay of cation-dependent DtxR function, and provides a level of quantitative analysis that cannot be obtained through more traditional assays (EMSA, DNase footprinting, equilibrium dialysis). It is useful for examining the ability of the regulatory circuit to control the expression of a desired gene.

4. Discussion

The systems described here demonstrate significant improvements on existing regulated expression systems which are dependent on the unique genetic, biophysical, and biochemical properties of metal dependent repressors and cognate operators. By developing an in vitro functional assay of DtxR-mediated repression one can compare the level of repression of a given gene of interest in vitro directly with in vivo findings.

Because both in vivo and in vitro assays developed here originate with plasmid DNA (pRS551-tacPtoxO-lacZ and pJL1, respectively), the operator sequence can also be easily changed by PCR (for slight changes) or restriction enzyme digestion and cloning (for larger alterations). This flexibility offers a relatively easy way to study the effect of operator sequence changes on DtxR-mediated repression.

### SUMMARY OF EXAMPLE 2

We have identified a gene in E. faecalis encoding a metal-dependent repressor, EfaR, with homology to the Corynebacterium diphtheriae toxin repressor, DtxR. The EfaR repressor regulates an operon isolated from E. faecalis acts as a potent promoter element for use in the expression of recombinant proteins in E. coli. Specific and metal ion-dependent interaction of EfaR with efaABC promoter/operator sequences is demonstrated by electrophoretic mobility shift analysis (EMSA). Furthermore, we demonstrate that EfaR, in the presence of metal ions, blocks transcription of an efaPO/lacZ transcriptional fusion reporter gene construct. The novel regulatory element and hybrids derived from it can be employed in the context of Example 1 and the claims of this specification.

### EXAMPLE 2

A putative adhesion involved in virulence of E. faecalis, EfaA, has been identified (Lowe, 1995) and proposed to be an important colonization factor in both endocarditis and urinary tract infections. EfaA and the genes in its operon, efaABC, are co-transcribed and encode the components of an ATP Binding Cassette (ABC) transporter. Evidence suggests that these transporters mediate high affinity uptake of Mn²⁺ (Jakubovics, 2000 and Novak, 1998). The structural gene encoding the DtxR-homologue from E. faecalis, efaR, was cloned and expressed in E. coli. The recombinant protein was purified to a greater than 98% homogeneity, and shown by EMSA to be a metal ion-dependent DNA binding protein, and to regulate expression of β-galactosidase from a efaABC promoter/operator lacZ transcriptional fusion analogous to manner in which DtxR regulates gene expression via toxP10 and the tacP/toxO element described above in EXAMPLE 1.

Methods

Bacterial strains, plasmids and media. Enterococcus faecalis ATCC# 29212 was purchased from Fisher Scientific. Escherichia coli strains and Enterococcus strains were maintained on Luria broth agar. Metal salts (MnCl₂, NiCl₂, Fe₂ (SO₄)₃, ZnCl₂) were added to M9 minimal medium to a final concentration of 0.1 μm. For maintenance of recombinant constructs in E. coli, ampicillin at 100 μg/ml, kananycin at 50 μg/ml or 25 μg/ml, and chloramphenicol at 14 μg/ml were used as needed.

Genetic Techniques

Restriction endonucleases were purchased from New England Biolabs (Beverly, Mass.). Chromosomal DNA from E. faecalis was isolated as described previously (Shankar, 1999). Plasmid DNA was isolated using a commercially available kit from Qiagen Inc. (Valencia, Calif.). Oligonucleotides were synthesized using Genelink (Hawthorne, N.Y.), Invitrogen (Carlsbad, Calif.), and Sigma Genosys (Woodlands, Tex.). The DNA open reading frame, efaR, encoding the putative DtxR-like protein was identified by in silico search of the unfinished genome of E. faecalis (www.tigr.com). The efaR gene was amplified from E. faecalis chromosomal DNA using PCR. Native Pfu polymerase Stratagene (La Jolla, Calif.) and primers (EFF GAAAGGATAGGATCCATGACACCA (SEQ ID NO:5) and EFR GCTACTTTTTCAAAGCTTAGTTTTCC (SEQ ID NO:6)) into which BamHI and HindIII sites (underlined) were incorporated to facilitate further in-frame fusion of the efaR gene. The PCR product was subcloned into BamHI- and Hindll-digested pQE-30 (Qiagen Inc.) and a modified pQE-30 termed, pQN, which lacks the 6× His-tag (SEQ ID NO:9). Nucleotide sequence analysis confirmed that the gene was fused in the correct reading frame.

Purification of Recombinant EfaR

The plasmid containing the efaR-coding region in pQN, termed pQN-efaR, was transformed into E. coli M15[pREP4] (Qiagen Inc.). The resulting recombinant strain was grown to early exponential phase and expression of the protein was induced with isopropyl-β-D-thiogalactoside (IPTG) at a final concentration of 1 mM and purified using standard methods.

Electrophoretic Mobility Shift Assays

Gel retardation assays were performed as described by Tao et al. (1992). A 106 bp PCR product (primers EFsitF and EFsitR) containing the 5′promoter/operator region of E. faecalis ABC operon was used as a DNA probe.

β-Galactosidase Promoter Fusion Construction

A DNA fragment containing the promoter/operator region and the ribosome binding site of the E. faecalis ABC transporter operon was amplified by PCR using primers (Efsit F GCGCCTAAGAATTCCTTTGCATTTTCTTAAA (SEQ ID NO:7) and Efsit R GAACAGCTAAGTGGATCCTTTTTCTCATGAA (SEQ ID NO:8)) yielding a 106 bp fragment engineered to contain an EcoRI and a BamHI restriction sites (underlined). The BamHI/EcoRI fragment was cloned into pRS551 (Boyd, 1990) creating the reporter fusion construct: pRS551-efaPO. Nucleotide sequence analysis confirmed that the promoter fusion was in the correct orientation.

β-Galactosidase Assays

Recombinant strains of E. coli containing the pQN-efa or empty pQN with the reporter plasmid pRS551-efaPO were grown in M9 alone, or M9 supplemented with the indicated metal ions at 0.1 μm final concentration for 12-16 hours. β-galactosidase activity was determined essentially as described by Miller (1972) and activity is reported in Miller Units.

Identification of DNA Encoding EfaR

Primers EFAF and EFAR were designed to facilitate cloning of a full length 659-base pair copy of the efaR gene into the pQE-30 expression vector. The nucleotide sequence of the cloned efaR gene was predicted to encode a 222 amino acid protein with a deduced molecular weight of 25.5 kDa. Alignment of the deduced amino acid sequence with other DtxR-like repressors indicates that EfaR is most closely related to SloR from S. mutans with 46% identity and 62% amino acid similarity. EfaR also has 39% identity and 56% similarity to SirR from Staphylococcus epidermidis and is 35% identical to and 55% similar to DtxR from C. diphtheriae (Boyd, 1990). Predictive structural algorithms (Protscale at www.expasy.org) suggest that EfaR contains a helix turn helix motif similar to that of DtxR, which has been shown to be involved in binding to the tox operator.

Expression and Analysis of the EfaR Protein in E. coli

The efaR gene was overexpressed in E. coli and was purified to near homogeneity, using methods described previously (Towe, 1992). Following purification, EfaR was shown by SDS-PAGE to migrate as a single protein band with a Mr of approximately 25 kDa.

Analysis of DNA Binding by EfaR

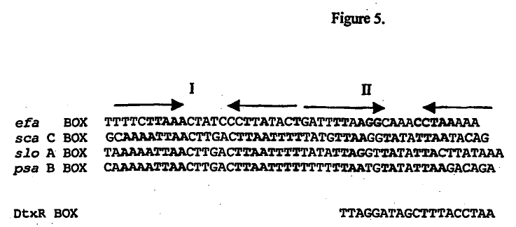

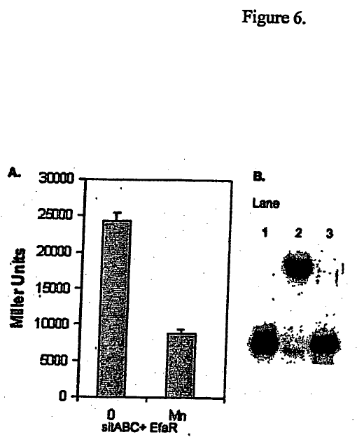

To determine if the EfaR protein regulates expression of the ABC operon in E. faecalis, we examined the region 5′ to the ABC operon for inverted repeats, which may serve as an EfaR binding site (FIG. 5). We found this region to contain two inverted repeat sequences, and therefore, used PCR to amplify this region. The PCR product was purified by gel electrophoresis and labeled with [³²P]. The oligonucleotide probe was then used in electrophoretic mobility shift assays with purified EfaR in the presence of poly dl/dC. As shown in FIG. 6B, EfaR was found to specifically bind to the probe in a metal ion-dependent fashion. Furthermore, the addition of polyclonal anti-EfaR antibodies to the reaction mixture resulted in a super-shift of the complex. Pre-immune sera did not result in a supershift (data not shown).

EfaR Represses Transcription of the Promoter Region of the Efa ABC Operon

The promoter/operator of the E. faecalis ABC transporter was subcloned immediately 5′ to the lacZYA creating the lacZ transcriptional fusion reporter plasmid pRS551-efaPO. Escherichia coli DH5αstrains carrying pRS551-efaPO and pQN-efaR were grown in M9 minimal medium supplemented with various transition metal ions. As shown in FIG. 6, lacZ expression was significantly repressed in recombinant E. coli grown in the presence of Mn²⁺ or Fe²⁺ (as determined by comparison of β-galactosidase activity by ANOVA with Dunn's correction M9 versus M9 plus Mn²⁺ p<0.01 or Fe²⁺ p<0.01 (FIG. 6A). When reporter strains of E. coli were grown in M9 minimal medium, lacZ was induced as shown by an increase in β-galactosidase levels. Thus EfaR is a metal ion-dependent repressor capable of regulating expression of genes in E. coli in a metal ion dependent fashion. EfaR target operator region in the efaABC box shares considerable homology with the corresponding promoter/operator elements in Staphylococcal species.

EfaR target operator region in the efaABC box shares considerable homology with the corresponding promoter/operator elements in Staphylococcal species

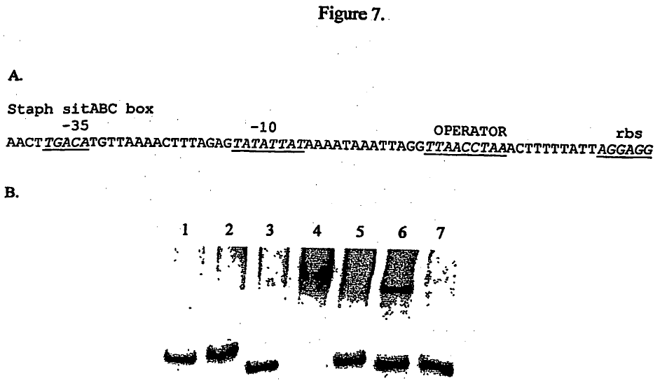

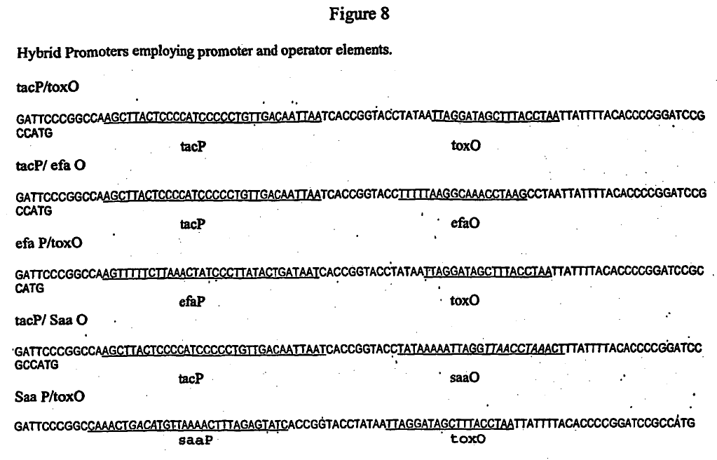

Unlike DtxR, the Enterococcal and Staphylococcal repressors appear to interact with larger, more complex DNA binding elements consisting of at least two inverted repeats (FIGS. 7A and B). Hybrid promoter elements employing the promoter and repressor elements of the efaABC and sitABC operons can also be employed in the construction of expression vectors. (FIG. 8.) We have synthesized two recombinant promoters using the tacP and efaO and the efaP and toxO. When used to drive lacZ expression the latter promoter expresses over 3000 MU of betagalactosidase in the presence of 2-2′dipyridyl.

### SUMMARY OF EXAMPLE 3

The invention is in the field of genetic engineering, specifically protein and RNA expression. The unwanted or mistimed expression of foreign proteins during the production of recombinant proteins in E. coli or other cell systems can have detrimental effects on the host organism. The majority of regulated gene expression systems which seek tightly control recombinant protein expression exhibit undesirable “leaky expression”. The present invention describes regulatory circuits and their methods of use to circumvent this problem using metal ion dependent repressor/operator tandems. Example 3 discloses methods of regulating the expression of DAB389 a diphtheria toxin based toxophore and DAB389IL-7 a IL-7 targeted fusion protein toxin.

### EXAMPLE 3

Interleukin 7 (IL-7) induces the proliferation of B cell progenitors and costimulates mature human T cell proliferation. IL-7 also induces cell growth in hematologic malignancies including acute lymphoblastic leukemia, chronic lymphocytic leukemia, acute myelogenous leukemia, and Sezary syndrome (vandeSpek et al,. 2002). This molecule comprised of the catalytic and transmembrane domains of diphtheria toxin (DAB389), fused to IL-7 is selectively cytotoxic for cells bearing the IL-7 receptor. Synthesis of DAB389 IL-7 in E. coli has been problematic. Over time selective pressure is placed upon clones which efficiently and appropriately express the desired molecule. We employed the ptacP/toxO expression vector to characterize the expression of DAB389 and DAB389-IL-7 in E. coli.

Expression of DAB389 in the ptacP/toxO Expression Vector System Using Variants of DtxR as Metal Ion Dependent Repressors

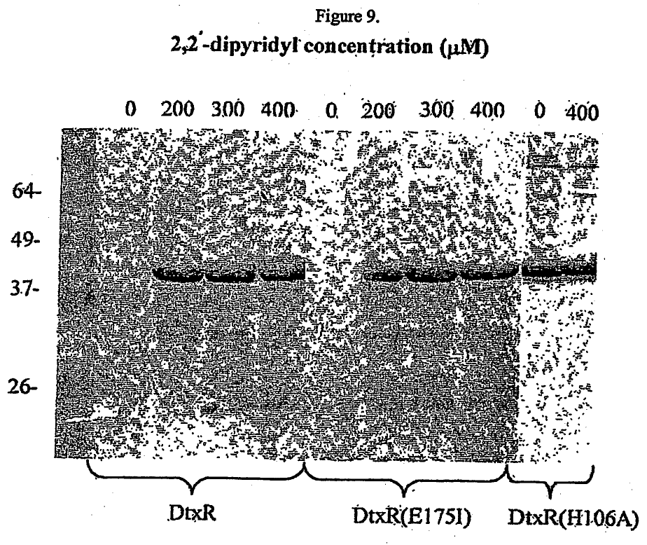

The expression of DAB389 was followed by Western analysis of protein extracts resolved on SDS PAGE gels. Expression was examined in the context of three different DtxR variants, (I) the wild type repressor, (2) a mutant H106A DtxR which destroys the metal ion dependent ability of the repressor and (3) DtxR E1751, a mutant which exhibits decreased sensitivity to the availability of activating metal ions. DAB389 expression was absent in complete cells grown in complete metal ion rich media using DtxR or DtxR E1751, however the mutant DtxR H106A was unable to repress DAB389 expression. [FIG 9.] Upon addition of increasing amounts of 2,2′-dipyridyl at concentrations from 200-400 uM expression of DAB389 was apparent regardless of the repressor variant employed. The ptacP/toxO expression vector system in conjunction with a functional metal ion dependent repressor was effective at suppression of DAB389 expression in E. coli.

Expression of DAB389IL-7 perm, a permutant of DAB389-IL-7, was followed from ptacP/toxO DAB389-IL-7 perm and pET DAB389-IL-7 perm by western analysis.

A ptacP/toxO DAB389-IL-7 perm expression vector was constructed and competent HMS174 (DE3) from Novagen were transformed with ptacP/toxO-DAB389-IL-7 perm or pET DAB389-IL-7 perm. Cultures were started from overnight grows and the OD₅₉₀ was measured approximately every 30 minutes. At OD₅₉₀=0.8, 1 uM IPTG or 300 uM D P was added to the ptacP/toxO DAB389-IL-7 cultures. At OD₅₉₀=0.575, 1 mM IPTG or 300 mM DP was added to the pET DAB389-IL-7 perm cultures. The cultures were grown at least an additional 2 hours with the OD₅₉₀ measurements being taken every 15-30 minutes.

Expression of DAB389-IL-7 from the ptacP/toxO promoter/operator occurred in the presence of DP but not I mM IPTG.

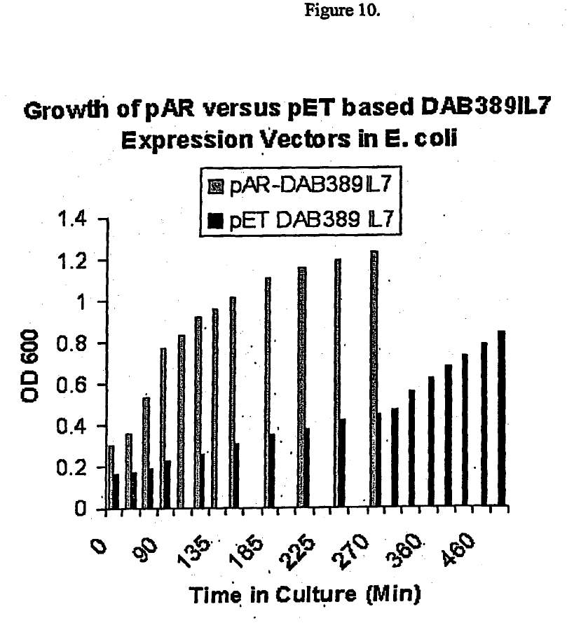

Expression of DAB389-IL-7 perm from the T7 promoter occurred in the presence of IPTG, DP and when nothing was added expression from the T7 promoter was greater than from the tacPtoxO promoter. Growth curves of ptacP/toxO DAB389-IL-7 perm and pET DAB389-IL-7 perm were also generated. (FIG. 10.) E. coli strain HMS174 (DE3) transformed with pET DAB389-IL-7 perm did not grow as rapidly as those transformed with ptacP/toxO-DAB389-IL-7 perm DtxR (E175I). Upon addition of the DP to the ptacP/toxO-DAB389-IL-7 perm DtxR (E1751) culture, bacterial growth slowed. Addition of 1 mM IPTG to either culture did not inhibit growth. The basal level of DAB389-IL-7 perm expression from the T7 promoter inhibited bacterial growth. The growth curve data agrees with the leaky expression of DAB389-IL-7 from pET vectors and confirms that DAB389-IL-7 expression inhibits the growth of the host bacteria. Continuous leaky expression of toxic proteins such as DAB389-IL-7 could result in dramatically lower yields of active protein. In contrast, expression of DAB389-IL-7 from metal ion regulated ptacP/toxO expression vector is effectively silenced until the time of induction thereby limiting the negative selective pressure on the recombinant expression strain.

### REFERENCES

1. Boyd, J., and J. R. Murphy. 1988. Analysis of the diphtheria tox promoter by site-directed mutagenesis. J. Bacteriol. 170(12):5949-5952.

2. Boyd, J., M. N. Oza, and J. R. Murphy. 1990. Molecular cloning and DNA sequence analysis of a diphtheria tox iron-dependent regulatory element (dtxR) from Corynebacterium diphtheriae. Proc. Natl. Acad. Sci. USA 87:5968-5972.

3. Chen, C. S., A. White, J. Love, J. R. Murphy, and D. Ringe. 2000. Methyl groups of thymine bases are important for nucleic acid recognition by DtxR. Biochem. 39:10397-10407.

4. Ding, X., H. Zeng, N. Schierring, D. Ringe, and J. R. Murphy. 1996. Identification of the primary metal ion-activation sites of the diphtheria tox repressor by X-ray crystallography and site directed mutational analysis. Nature Struct. Biol. 3(4):382-387.

5. Hill, P. J., A. Cockayne, P. Landers, J. A. Morrissey, C. M. Sims, and P. Williams. 1998. SirR, a novel iron-dependent repressor in Staphylococcus epidermidis. Infect. Immun. 66(9):4123-4129.

6. Hudson, M. C., Stewart, G. C., 1986. Differential utilization of Staphylococcus aureus promoter sequences by Escherichia coli and Bacillus subtilis. Gene 48, 93-100.

7. Hung, A., Thillet, J., Pictet, R., 1989. In vivo selected promoter and ribosome binding site up-mutations: demonstration that the Escherichia coli bla promoter and a Shine-Dalgamo region with low complementarity to the 16S ribosomal RNA function in Bacillus subtilis. Molec. Gen. Genet. 219, 129-136.

8 Jakubovics, N. S., A. W. Smith, and H. F. Jenkinson, 2000. Expression of the virulence-related Sca (Mn2+)permease in Streptococcus gordonii is regulated by a diphtheria toxin metallorepressor-like protein ScaR. Mol. Microbiol. 38(1): 140-153.

9. Kitten, T., C. L. Munro, and S. M. Michalek, 2000. Genetic characterization of a Streptococcus mutans LraI family operon and role in virulence. Infect. Immun. 68(8):4441-4451.

10. Kolenbrander, P. E., R. N. Andersen, and N. Ganeshkumar. 1994. Nucleotide sequence of the Streptococcus gordonii PK488 co-aggregation adhesion gene, scaA, and ATP-binding cassette. Infect. Immun. 62:4469-4480.

11. Laemli, U. K. 1970. Cleavage of structural proteins during the assembly of bacteriophage T4. Nature (London) 227:159-176.

12. Love J F, Murphy J R. 2002 Design and development of a novel genetic probe for the analysis of repressor-operator interactions. J Microbiol Methods. September 51 (1):63-72.

13 Lowe, A. M., P. A. Lambert, and A. W. Smith, 1995. Cloning of an Enterococcus faecalis endocarditis antigen: homology with adhesions from some oral streptococci. Infect. Immun. 63(2):703-706.

14. Maniatis, T., Fritsch, E. F., Sambrook, J., 1982. Isolation of Bacteriophage λ In: Molecular Cloning: A Laboratory Manual, Cold Spring Harbor Laboratory, Cold Spring Harbor, New York, pp. 76-85.

15. Miller, J. H., 1977. Assay of β-galactosidase. In: Experiments in Molecular Genetics, Cold Spring Harbor Laboratory, Cold Spring Harbor, New York, pp. 352-355.

16. Murray, B. E. 2000. Vancomycin-resistant enterococcal infections. New. Engl. J. Med. 342(10):710-721.

17. Novak, R., J. S. Braun, E. Charpentier, and E. Tuomanen. 1998. Penicillin tolerance genes of Streptococcus pneumoniae: the ABC-type manganese permease complex, Psa. Mol. Microbiol. 29:1285-1296.

18. Oguiza, J.A., Tao, X., Marcos, A.T., Martin, J. F.,. Murphy, J. R., 1995. Molecular cloning, DNA sequence analysis, and characterization of the Corynebacterium diphtheriae dtxR homolog from Brevibacterium lactofermentum. J. Bac. 177, 465-467.

19. Pohl, E., Holmes, R. K., Hol, W. G. J., 1999. Crystal structure of a cobalt-activated diphtheria toxin repressor-DNA complex reveals a metal-binding SH3-like domain. J. Molec. Biol., 292, 653-667.

20. Posey, J. E., J. M. Hardham, S. J. Norris, and F. C. Gherardini, 1999. Characterization of a manganese-dependent regulatory protein, TroR from Treponema pallidum. Proc. Natl. Acad. Sci. USA 96:10887-10892.

21. Que, Q., Helmann, J. D., 2000. Manganese homeostasis in Bacillus subtilis is regulated by MntR, a bifunctional regulator related to the diphtheria toxin repressor family of proteins. Molec. Microbiol. 35, 1454-1468.

22. Qui, X., E. Pohl, R. K. Holmes, and W. G. J. Hol, 1996. High-resolution structure of the diphtheria toxin repressor complexed with cobalt and manganese reveals an SH3-like third domain and suggests a possible role of phosphate as co-repressor. Biochem. 35:12292-12302.

23. Qui, X., C. L. Verlinde, S. Zhang, M. P. Schmitt, R. K. Holmes, and W. G. J. Hol, 1995. Three dimensional structure of the diphtheria toxin repressor in complex with divalent cation co-repressor. Structure 3:87-100.

24. Schiering, N., X. Tao, H. Zeng, J. R. Murphy, G. A. Petsko, and D. Ringe, 1995. Structures of the apo- and the metal ion-activated forms of the diptheria tox repressor from Corynebacterium diphtheriae. Proc. Natl. Acad. Sci. USA 92:9843-9850.

25. Schmitt, M. P., M. Predich, L. Doukan, I. Smith, and R. K. Holmes. 1995. Characterization of an iron-dependent regulatory protein (IdeR) of Mycobacterium tuberculosis as a functional homolog of the diphtheria toxin repressor (DtxR) from Corynebacterium diphtheriae. Infect. Immun. 63:4284-4289.

26. Schmitt, M. P., 1997. Transcription of the Corynebacterium diphtheriae hmuO gene is regulated by iron and heme. Infect. Immun. 65, 4634-4641.

27. Schmitt, M. P. Holmes, R. K., 1993. Analysis of diphtheria toxin repressor-operator interactions and characterization of mutant repressor with decreased binding activity for divalent metals. Molec. Microbiol. 9, 173-181.

28. Schmitt, M. P., Holmes, R. K., 1994. Cloning, sequence, and footprint analysis of two promoter/operators from Corynebacterium diphtheriae that are regulated by the Diphtheria Toxin Repressor (DtxR) and iron. J. Bac. 176, 1141-1149.

29. Shankar, V., A. S. Bagdayan, M. M. Huycke, G. Lindahl, and M. S. Gilmore. 1999. Infection-derived Enterococcus faecalis strains are enriched in esp, a gene encoding a novel surface protein. Infect. Immun. 67(1): 193-200.

30. Spatafora, G., M. Moore, S. Landgren, E. Stonehouse, and S. Michalek. 2001. Expression of Streptococcus mutans fimA is iron-responsive and regulated by a DtxR homologue. Microbiol. 17:1599-1610.

31. Tao, X. Murphy, J. R., 1992. Binding of the metalloregulatory protein DtxR to the diphtheria tox operator requires a divalent heavy metal ion and protects the palindromic sequence from DNase I digestion. J. Biol. Chem. 267, 21761-21764.

32. Tao, X., J. Boyd, and J. R. Murphy. 1992. Specific binding of the diphtheria tox regulatory element DtxR to the tox operator requires divalent heavy ions and a 9-base-pair interrupted palindromic sequence. Proc. Natl. Acad. Sci. USA 89:5897-5901.

33. Tao, X, Murphy, J.R., 1993. Cysteine-102 is positioned in the metal binding activation site of the Corynebacterium diphtheriae regulatory element DtxR. Proc. Natl. Acad. Sci. USA, 90, 8524-8528.

34. Tao, X., and J. R. Murphy. 1994. Determination of the minimal essential nucleotide sequence for diphtheria tox repressor binding by in vitro affinity selection. Proc. Natl. Acad. Sci. USA 91:9646-9650.

35. Tao, X., H. Y. Zeng, and J. R. Murphy. 1995. Transition metal ion activation of DNA binding by the diphtheria tox repressor requires the formation of stable homodimers. Proc. Natl. Acad. Sci. USA 92:6803-6807.

36. Towbin, H., T. Staehelin, and J. Gordon. 1979. Electrophoretic transfer of proteins from polyacrylamide gels to nitrocellulose sheets: Procedure and some applications. Proc. Natl. Acad. Sci. USA 76:4350-4354.

37. Twigg, P. D., Parthasarathy, G., Guerrero, L., Logan. T. M., Caspar, L. D. 2001. Disordered to ordered folding in the regulation of diphtheria toxin repressor activity. Proc. Natl. Acad. Sci. USA 98(20):11259-11264.

38. vanderSpek J C, Sutherland J A, Gill B M, Gorgun G, Foss FM, Murphy J R. Structure function analysis of interleukin 7: requirement for an aromatic ring at position 143 of helix D. Cytokine. 2002 17(5):227-33.

39. Wang, G., G. P. Wylie, P. D. Twigg, D. L. D. Caspar, J. R. Murphy, and T. M. Logan. 1999. Solution structure and peptide binding studies of the C-terminal Src homology 3-like domain of the diphtheria toxin repressor protein. Proc. Natl. Acad. Sci. USA 96:6119-6124.

40. White, A., X. Ding, J. C. vanderSpek, J. R. Murphy, and D. Ringe. 1998. Structure of the metal-ion-activated diphtheria toxin repressor/tox operator complex. Nature 394:502-506.

### INDUSTRIAL APPLICABILITY

The present invention is useful in the production of proteins for use in a variety of industries such as pharmaceutics, food and agriculture.

All publications cited in the specification including websites, are indicative of the level of skill of those skilled in the art to which this invention pertains. All these publications are herein incorporated by reference to the same extent as if each individual publication were specifically and individually indicated to be incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A composition of matter, comprising:
a first nucleic acid encoding a repressor protein activated by a metal ion activator, and a second nucleic acid comprising a promoter, an operator sequence and at least one structural gene in operable association with each other, such that in the presence of the metal ion, activated repressor binds said operator and inhibits expression of said structural gene.

2. The composition of claim 1, which is a recombinant bacterium.

3. The composition of claim 2, wherein said bacterium is E. coli.

4. The composition of claim 2, wherein said first nucleic acid is contained in genome of said bacterium in operable association with a promoter native to said bacterium, and said second nucleic acid is contained in non-chromosomal nucleic acid.

5. The composition of claim 4, wherein said non-chromosomal nucleic acid comprises a plasmid.

6. The composition of claim 2, further comprising a growth medium.

7. The composition of claim 6, further comprising the metal ion activator of said repressor protein.

8. The composition of claim 1, further comprising a first vector comprising said first nucleic acid, and a second vector comprising said second nucleic acid.

9. The composition of matter of claim 1, further comprising the at least one structural gene.

10. A composition of matter, comprising: a first nucleic acid encoding a repressor protein activated by a metal ion activator, and a second nucleic acid comprising a promoter, an operator sequence and a multiple cloning site for introduction of at least one structural gene, each in operable association with each other, such that in the presence of the metal ion, activated repressor binds said operator and inhibits expression of the at least one structural gene.

11. A composition, comprising: an E. coli cell comprising a first non-native nucleic acid encoding DtxR or a mutant or a homolog thereof, and a second nucleic acid comprising a promoter, an operator that binds said DtxR or mutant or homolog thereof, and at least one structural gene, in operable association with each other such that in the presence of a metal ion activator, said DtxR or mutant or homolog thereof is activated and binds said operator and inhibits expression of said at least one structural gene.

12. The composition of claim 11, further comprising a growth medium.

13. The composition of claim 11, further comprising 2,2′-dipyridyl.

14. The composition of claim 11, wherein said promoter comprises a T7 promoter.

15. The composition of claim 11, wherein said promoter comprises TacP.

16. The composition of claim 11, wherein said promoter comprises EfaP.

17. The composition of claim 11, wherein said promoter comprises MntP.

18. The composition of claim 11, wherein said operator comprises toxO.

19. The composition of claim 18, wherein said repressor comprises a homolog of DtxR which is IdeR.

20. The composition of claim 18, wherein said repressor comprises a mutant of DtxR which is E175K or E175I.

21. The composition of claim 11, wherein said operator comprises efaO.

22. The composition of claim 21, wherein said repressor comprises a homolog of DtxR which is EfaR.

23. The composition of claim 11, wherein said operator comprises mntO.

24. The composition of claim 23, wherein said first nucleic acid encodes a homolog of DtxR which is MntR or SirR.

25. The composition of claim 11, wherein said promoter and operator together are represented by the sequence: TTTTCTTAAACTATCCCTTATACTGATTTTAAGGCAAACCTAAAAA (SEQ ID NO:13).

26. A method for producing a protein, comprising:
(a) transforming a prokaryotic cell with a first nucleic acid encoding a repressor protein, and a second nucleic acid comprising a second nucleic acid comprising a promoter, an operator and a structural gene encoding the protein, each in operable association with each other;
(b) growing transformed prokaryotic cells of (a) in a medium comprising a metal ion activator of the repressor, wherein activated repressor binds the operator;
(c) reducing activity of the activator to cause expression of the structural gene; and
(d) isolating the protein from the prokaryotic cell or the medium.

27. The method of claim 26, wherein said cells comprise E. coli cells.

28. The method of claim 26, wherein said first nucleic acid encodes DtxR or a homolog thereof that binds the operator.

29. The method of claim 26, wherein said metal ion activator comprises Fe++ions.

30. The method of claim 26, wherein said reducing comprises adding a chelator of the metal ions to the growth medium.

31. A method for producing a protein, comprising:
(a) transforming an E. coli cell with a first nucleic acid encoding a metal ion-dependent repressor which is DtxR or a homolog thereof, and a second nucleic acid comprising a promoter, an operator that binds the DtxR or homolog thereof, and a structural gene encoding the protein, each in operable association with each other;
(b) growing transformed cells of (a) in a medium comprising a metal ion that activates the repressor, wherein activated repressor binds the operator;
(c) reducing free metal ion in the medium to cause expression the structural gene; and
(d) isolating the protein from the E. coli or the medium.

32. The method of claim 31, wherein said reducing comprises adding 2,2′-dipyridyl to the medium.

33. The method of claim 32, wherein the 2,2′-dipyridyl is added to the medium in an amount of from about 100 μM to about 300 μM.

34. The method of claim 32, wherein the 2,2′-dipyridyl is added to the medium at a time when the medium has an optical density of from about 0.4 to about 0.8, measured in terms of OD₅₀₀.

35. A method for producing a protein, comprising:
preparing a system comprising a first nucleic acid encoding a repressor protein, and a second nucleic acid comprising a second nucleic acid comprising a promoter, an operator and a structural gene encoding the protein, each in operable association with each other; wherein said system further comprises a metal ion activator of the repressor, wherein activated repressor binds the operator; and
reducing activity of the activator to cause expression of the structural gene.


SEQUENCE LISTING (25 sequences)

SEQ ID NO: 1 - DNA, 27 bp, Corynebacterium diphtheriae
ataattaggatagctttacctaattat

SEQ ID NO: 2 - DNA, 19 bp, Corynebacterium diphtheriae
gtaggttaggctaacctat

SEQ ID NO: 3 - DNA, 25 bp, Artificial Sequence
  Description of Artificial Sequence Synthetic consensus oligonucleotide
ananttaggntagnctannctnnnn

SEQ ID NO: 4 - DNA, 19 bp, Artificial Sequence
  Description of Artificial Sequence Synthetic degenerate oligonucleotide
twaggttagsctaacctwa

SEQ ID NO: 5 - DNA, 24 bp, Artificial Sequence
  Description of Artificial Sequence Synthetic primer
gaaaggataggatccatgacacca

SEQ ID NO: 6 - DNA, 26 bp, Artificial Sequence
  Description of Artificial Sequence Synthetic primer
gctactttttcaaagcttagttttcc

SEQ ID NO: 7 - DNA, 31 bp, Artificial Sequence
  Description of Artificial Sequence Synthetic primer
gcgcctaagaattcctttgcattttcttaaa

SEQ ID NO: 8 - DNA, 31 bp, Artificial Sequence
  Description of Artificial Sequence Synthetic primer
gaacagctaagtggatcctttttctcatgaa

SEQ ID NO: 9 - protein, 6 aa, Artificial Sequence
  Description of Artificial Sequence Synthetic 6x His tag
HHHHHH

SEQ ID NO: 10 - DNA, 63 bp, Corynebacterium diphtheriae
ccctgttgacaattaatcatcggctcgtataatgtgtggaattgtgagcggataacaatttca

SEQ ID NO: 11 - DNA, 57 bp, Corynebacterium diphtheriae
gcattgatttcagagcacccttataattaggatagctttacctaattattttatgag

SEQ ID NO: 12 - DNA, 64 bp, Artificial Sequence
  Description of Artificial Sequence Synthetic construct of tacPtoxO promoter-operator
ccctgttgacaattaatcaccggtacctataattaggatagctttacctaattattttacaccc

SEQ ID NO: 13 - DNA, 46 bp, Enterococcus faecalis
ttttcttaaactatcccttatactgattttaaggcaaacctaaaaa

SEQ ID NO: 14 - DNA, 47 bp, Streptococcus gordonii
gcaaaattaacttgacttaatttttatgttaaggtatattaatacag

SEQ ID NO: 15 - DNA, 49 bp, Streptococcus mutans
taaaaattaacttgacttaatttttatattaggttatattacttataaa

SEQ ID NO: 16 - DNA, 48 bp, Streptococcus pneumoniae
caaaaattaacttgacttaattttttttttaatgtatattaagacaga

SEQ ID NO: 17 - DNA, 19 bp, Artificial Sequence
  Description of Artificial Sequence Synthetic consensus oligonucleotide
ttaggatagctttacctaa

SEQ ID NO: 18 - DNA, 25 bp, Enterococcus faecalis
ttttcttaaactatcccttatactg

SEQ ID NO: 19 - DNA, 20 bp, Enterococcus faecalis
ttttaaggcaaacctaaaaa

SEQ ID NO: 20 - DNA, 71 bp, Staphylococcus aureus
aacttgacatgttaaaactttagagtatattataaaataaattaggttaacctaaactttttattaggagg

SEQ ID NO: 21 - DNA, 105 bp, Artificial Sequence
  Description of Artificial Sequence Synthetic hybrid promoter
gattcccggccaagcttactccccatccccctgttgacaattaatcaccggtacctataattaggatagctttacctaattattttacaccccggatccgccatg

SEQ ID NO: 22 - DNA, 105 bp, Artificial Sequence
  Description of Artificial Sequence Synthetic hybrid promoter
gattcccggccaagcttactccccatccccctgttgacaattaatcaccggtacctttttaaggcaaacctaagcctaattattttacaccccggatccgccatg

SEQ ID NO: 23 - DNA, 105 bp, Artificial Sequence
  Description of Artificial Sequence Synthetic hybrid promoter
gattcccggccaagtttttcttaaactatcccttatactgataatcaccggtacctataattaggatagctttacctaattattttacaccccggatccgccatg

SEQ ID NO: 24 - DNA, 106 bp, Artificial Sequence
  Description of Artificial Sequence Synthetic hybrid promoter
gattcccggccaagcttactccccatccccctgttgacaattaatcaccggtacctataaaaattaggttaacctaaactttattttacaccccggatccgccatg

SEQ ID NO: 25 - DNA, 99 bp, Artificial Sequence
  Description of Artificial Sequence Synthetic hybrid promoter
gattcccggccaaactgacatgttaaaactttagagtatcaccggtacctataattaggatagctttacctaattattttacaccccggatccgccatg